SEQUENCE LISTING (2 sequences)

SEQ ID NO: 1 - DNA, 19 bp, Artificial Sequence
  primer
agatttggacctgcgagcg

SEQ ID NO: 2 - DNA, 18 bp, Artificial Sequence
  primer
gaagccggggcaactcac

(19) United States
(12) Patent Application Publication
(10) Pub. No.: US 2022/0282331 A1
(43) Pub. Date: Sep. 8, 2022

(54) EPIGENETIC SIGNATURE OF ENDOMETRIOSIS ON THE BASIS OF ACELLULAR DNA

(71) Applicant: ENDOLIFE, PARIS (FR)

(72) Inventors: André HAZOUT, PARIS (FR); Moncef BEN KHALIFA, LE MESNIL-SAINT-DENIS (FR)

(21) Appl. No.: 17/624,768

(22) PCT Filed: Jul. 6, 2020
(86) PCT No.: PCT/EP2020/069015

§ 371 Date: Jan. 4, 2022

(30) Foreign Application Priority Data
Jul. 5, 2019 (EP) 19305920.1

(51) Int. Cl. C12Q 1/6883; G01N 33/53; G16B 45/00

(52) U.S. Cl. CPC C12Q 1/6883; G01N 33/5308; G16B 45/00; C12Q 2600/154; G01N 2800/364

(57) ABSTRACT

Disclosed is a method for non-invasive diagnosis of endometriosis on the basis of the measurement of the level of acellular DNA in a biological sample from an individual, wherein a measured level of acellular DNA lower than the reference threshold can be used to rule out an endometriosis diagnosis. When the measured level of acellular DNA is greater than the reference threshold, a step of measuring the methylation level of at least 15 genes involved in endometriosis enables the diagnosis of endometriosis to be made in the patient tested.

### FIELD OF THE INVENTION

The present invention relates to the field of non-invasive diagnosis of endometriosis. More particularly, the invention proposes steric markers (level of acellular DNA and epigenetic markers) making it possible to screen for endometriosis and to characterize its intensity.

### TECHNICAL BACKGROUND

Endometriosis is a chronic estrogen-dependent inflammatory disease caused by migration of cells from the endometrium to outside of the uterine cavity, mainly on the pelvic organs and tissues. This inflammatory state associated with pelvic pains and, sometimes, a state of infertility, afflicts 3 to 10% of young women of childbearing age.

The disease is heterogeneous, ranging from superficial peritoneal and serous lesions to endometriotic cysts in the ovaries (endometrioma), nodules in deep endometriosis, and can often be accompanied by fibrosis and adhesions. Painful endometriosis can occur in young prepubescent girls (Marsh and Laufer, 2005) then from puberty to menopause, where menstrual cycles and pain disappear.

We know that the growth of ectopic endometrial cells is dependent on estrogens. Endometriosis is widely perceived as a regurgitation of menstrual blood with migration of endometrial cells to all the surrounding organs and sometimes beyond: abdomen, lungs, brain and elsewhere.

Very rarely, endometriosis has also been seen in the male genitourinary tract (Beckman et al., 1985; Fukunaga, 2012; Rei and Feloney, 2018). Twenty-two cases of hepatic endometriosis have also been published (Liu et al., 2015). This is an argument against the predominant theory of retrograde flow as it has been studied in female endometriosis. It is essential to explain why only 10% of women develop endometriosis whereas retrograde menstruation occurs in 76% to 90% of women of childbearing age (Blumenkrantz et al., 1981; Halme et al., 1984).

The diagnosis of endometriosis is made during a laparoscopy and confirmed by analysis of the lesions extracted during a laparoscopy operation. The treatment is often medical for mild to moderate forms (stages I and II) and surgical, immediately or after hormone therapy, for severe forms (stages III and IV).

Endometriosis has variously been described as a hormonal or immune disease, and a genetic disease triggered by exposure to environmental factors. In addition, many studies have suggested various epigenetic aberrations in the pathogenesis of endometriosis.

The heterogeneity of the phenotype of the disease is authenticated by a large number of false negative laparoscopies among symptomatic women. Moreover anatomopathological, immunohistochemical and epigenetic examinations of lesions have not proved to be reliable (Soo Hyun Ahn et al., 2017), in particular concerning methylation of the genomic DNA of the progesterone receptor B, e-cadherin, homeobox A10 (HOXA10), estrogen receptor beta, steroidogenic factor 1(SF1) and aromatase.

The aberrant expression of DNA methyltransferase, which adds a methyl group in position 5 of the cytosine bases in the CpG (Cytosine phosphate Guanine) island of the gene promoter, silencing the corresponding genetic expression, has been demonstrated in endometriosis (Nasu et al., 2011).

In the human blood, the presence of free-circulating cell free DNA (cfDNA) was reported in 1948 by Mendel and Metais (Mandel and Metais, 1948). cfDNA has been studied under a wide range of physiological and pathological conditions, in particular inflammatory disorders, oxidative stress, infertility of couples (EP2879696B1) and malignant tumors.

In healthy individuals, during phagocytosis, the apoptotic or necrotic bodies are ingested by macrophages. Hence, the phenomenon of free DNA release cannot occur. On the other hand, when DNA fragments remain in the nucleosomes which are released by the macrophages, they are protected from enzymatic degradation and thus remain in the bloodstream.

cfDNA is composed of double-stranded nucleic acids with lower molecular weight than genomic DNA. The size of these genomic fragments is variable, ranging from 70 to 200 pb for the shortest and up to 21 kb for the longest. cfDNA is present in healthy patients at blood concentrations evaluated at between 50 and 250 ng/ml (EP2879696B1).

The biological mechanisms by which free DNA is released into the blood are not entirely understood. Fragments of free DNA can originate from necrotic cells engulfed by macrophages which are then partially released. According to this hypothesis, the level of cfDNA should be correlated with the extent of the cellular necrosis and/or apoptosis.

The clearance of the cfDNA from the bloodstream is rapid (half-life: 16.3 min.). It is known that cfDNA is sensitive to plasma nucleases, but renal and hepatic clearance are also involved in its removal.

cfDNA can be isolated from the plasma and from the serum, but the serum has a DNA concentration that is around six times greater (incorporation in the cells).

DNA levels and fragmentation patterns offer interesting possibilities for diagnostic and prognostic purposes.

Currently, only the study by Zachariah (2009) has demonstrated significantly higher nuclear and mitochondrial cfDNA in a group of women suffering from minimal to mild endometriosis than in a control group (p=0,046). The threshold from an ROC curve demonstrated a sensitivity of 70% and an 87% specificity. The author concluded that the circulating cfDNA could constitute a potential biomarker for minimal and mild endometriosis.

### SUMMARY OF THE INVENTION

The present invention relates to a non-invasive (in vitro) screening method for endometriosis, based on measuring the level of acellular DNA in a biological sample from an individual, followed, when the acellular DNA level is greater than a reference threshold, by measuring the level of methylation of at least 15 genes involved in endometriosis. A measured level of acellular DNA less than a reference threshold (identical to or different from the first) allows the possibility of endometriosis to be ruled out.

According to the invention, (i) a hypermethylation of genes selected among CALD1, RRP1, FN1, FAM87B, TCEAL6, RPL29P2, ATP11A-AS1, DIP2C, SLCO2B1, RMI2, MIR3170, LINC01007, TSPAN17, MIR4693, HYOU1, TLR4, ADGRL3, IL6, VIRMA, MKRN1, INSIG1, ROR2, MRPL3, FMNL2, TMEM19, ZNF438, LINC01192, RCBTB1, TSPAN33, NKD2, FGFR2, TPRG1, MIR4644, FOXO4, FSTL1 and CLMN, and/or (ii) a hypomethylation of genes selected among NT5C2, NAV1, SOD3, C3, UBE3A, MIR4655, MYO5C, COX6C, MIR6133, BRSK2, MIR4277, MIR4251, MN1, MIR3666, AZIN1, MIR4251, SLC37A2, FZD10, STAU2-AS1, TDRDS, USP1, ACVR2A, FBXO38, FASN, MKRN9P and PCCA-AS1, constitute markers of an endometriosis, the potential development of which is increasingly significant the larger the hyper- or hypomethylation measured.

The present invention also relates to kits for implementing the methods of the invention.

### DESCRIPTION OF THE FIGURES

FIG. 1: gene network created by Ingenuity Pathway Analysis (IPA), obtained only from hypermethylated genes in endometriotic women. The solid lines between the nodes indicate a direct molecular interaction between the connected genes, whereas the dashed lines indicate an indirect functional interaction between the genes. FIGS. 4 and 5 by considering all the differentially methylated genes (DMG: hyper- and hypomethylated).

FIG. 2: Transmembrane conduction paths: receptor for TGF beta and FRIZZLED proteins.

FIG. 3: gene network created by Ingenuity Pathway Analysis (IPA), obtained only from hypomethylated genes in endometriotic women. The solid lines between the nodes indicate a direct molecular interaction between the connected genes, whereas the dashed lines indicate an indirect functional interaction between the genes.

FIG. 4: gene network created by Ingenuity Pathway Analysis (IPA), obtained by considering all the differentially methylated genes (DMG: hyper- and hypomethylated). The solid lines between the nodes indicate a direct molecular interaction between the connected genes, whereas the dashed lines indicate an indirect functional interaction between the genes.

FIG. 5: gene network created by Ingenuity Pathway Analysis (IPA), obtained by considering all DMG (hyper- and hypomethylated). The solid lines between the nodes indicate a direct molecular interaction between the connected genes, whereas the dashed lines indicate an indirect functional interaction between the genes.

### DETAILED DESCRIPTION

According to a first aspect, the present invention relates to an in vitro screening method for endometriosis, comprising

(i) measuring the level of acellular DNA in a biological sample from an individual, and

(ii) comparing the level of acellular DNA with a predetermined threshold,

wherein a level of acellular DNA greater than the predetermined threshold indicates that the individual may have endometriosis, and a measured level of acellular DNA less than the predetermined threshold allows active endometriosis to be ruled out

This method can be performed in order to establish, in a non-invasive manner, an endometriosis diagnosis in a human, in particular in a woman, in particular in a woman of childbearing age.

According to a particular embodiment of the above method, the sample for which the level of acellular DNA is measured is a sample of biological fluid. Biological fluid that can be used in the context of the invention includes, but is not limited to, blood, plasma and serum.

A person skilled in the art is able, based on his/her general knowledge, to adjust the threshold to which the individual's acellular DNA will be compared, depending on the biological fluid in which this level is measured, the technology used for this measurement and other parameters connected to the clinical profile of the individual. For this purpose, a person skilled in the art can, for example, by performing routine tasks, carry out measurements of the level of acellular DNA in one or more cohorts of patients suffering from endometriosis and in groups of control individuals (for example, women not having endometriosis). A person skilled in the art will then use, for example, the Receiver Operating Characteristic, or “ROC curve” technique, frequently used in clinical biology, in order to choose one or more reference values according to the needs (such as to favor the sensitivity or specificity of the test).

The quantification of the cfDNA can be carried out as indicated in the experimental part below, or using other methods described in the scientific literature, in particular fluorometric or spectrophotometric methods such as QUBIT® (Life Technologies) or NANODROP™ (Thermo Scientific). Recently, the analysis of acellular DNA has been widely described in methods for diagnosis of certain cancers or in the prenatal diagnosis of chromosomal anomalies. Several technologies for isolating and analyzing acellular DNA have also been described, both in scientific publications as well as in the patent literature. A person skilled in the art can ideally choose, from the multiple technologies described, that which appears appropriate for implementing the invention.

When the measured level of acellular DNA is greater than a predetermined threshold, the method of the invention also comprises a step (iii) of determining the level of methylation of certain genes involved in endometriosis, still from the acellular DNA present in a serum sample of the individual. In a preferred embodiment of the invention, this step comprises analyzing the methylation of at least 15 genes selected from among the genes described in the table below:

TABLE 1
genes having a different methylation profile (hyper- or
hypomethylation) in endometriotic patients

| | GenBank | | GenBank | | GenBank |
|---|---|---|---|---|---|
| Name | identifier | Name | identifier | Name | identifier |
| CALD1 | NM_033140 | ROR2 | NM_004560 | MYO5C | NM_018728 |
| RRP1 | NM_003683 | MRPL3 | NM_007208 | COX6C | NM_004374 |
| FN1 | NM_212482 | FMNL2 | NM_052905 | MIR6133 | NR_106749 |
| FAM87B | NR_103536 | TMEM19 | NM_018279 | BRSK2 | NM_001256629 |
| TCEAL6 | NM_001006938 | ZNF438 | NM_182755 | MIR4277 | NR_036240 |
| RPL29P2 | NR_002778 | LINC01192 | NR_033945 | MIR4251 | NR_036215 |
| ATP11A-AS1 | NR_046661 | RCBTB1 | NM_018191 | MN1 | NM_002430 |
| DIP2C | NM_014974 | TSPAN33 | NM_178562 | MIR3666 | NR_037439 |
| SLCO2B1 | NM_007256 | NKD2 | NM_001271082 | AZIN1 | NM_148174 |
| RMI2 | NM_152308 | FGFR2 | NR_073009 | MIR4251 | NR_036215 |
| MIR3170 | NR_036129 | TPRG1 | NM_198485 | SLC37A2 | NM_198277 |
| LINC01007 | NR_103749 | MIR4644 | NR_039787 | FZD10 | NM_007197 |
| TSPAN17 | NM_012171 | FOXO4 | NM_005938 | STAU2-AS1 | NR_038406 |
| MIR4693 | NR_039842 | FSTL1 | NM_007085 | TDRD5 | NM_001199091 |
| HYOU1 | NM_006389 | CLMN | NM_024734 | USP1 | NM_003368 |
| TLR4 | NM_138554 | NT5C2 | NM_012229 | ACVR2A | NM_001616 |
| ADGRL3 | NM_015236 | NAV1 | NM_020443 | FBXO38 | NM_001271723 |
| IL6 | NM_000600 | SOD3 | NM_003102 | FASN | NM_004104 |
| VIRMA | NM_015496 | C3 | NM_000064 | MKRN9P | NR_033410 |
| MKRN1 | NR_117084 | UBE3A | NM_130839 | PCCA-AS1 | NR_047686 |
| INSIG1 | NM_198337 | MIR4655 | NR_039799 | | |

The methylation profile obtained in step (iii) is then compared with one or more reference profiles in order to obtain a differential profile, the analysis of which makes it possible to determine the presence or absence of endometriosis.

With regard to the reference profile or profiles, it is obvious that a person skilled in the art can easily, through routine tasks, measure the level of methylation of all or part of the genes mentioned above in various cohorts (patients having or not having endometriosis) and thus establish, depending on the technology used for the measurement of these methylation levels, reference profiles to which the profile of the patient will be compared. If necessary, a person skilled in the art can establish profiles corresponding to different forms of endometriosis, by carrying out these routine measurements on different cohorts of patients presenting more or less severe forms of endometriosis. The profile of the patient will then be compared to these different profiles, which will enable not only the diagnosis of endometriosis to be established, but also a determination to be made of the intensity or severity.

During this analysis, a hypermethylation or a hypomethylation of these genes with respect to the level of methylation of the same genes in a population of non-endometriotic individuals will be considered, for example, to constitute a sign suggestive of endometriosis or a marker of endometriosis. More precisely, the methylation profiles below are considered as signs suggestive of endometriosis:

(i) a hypermethylation of genes selected among CALD1, RRP1, FN1, FAM87B, TCEAL6, RPL29P2, ATP11A-AS1, DIP2C, SLCO2B1, RMI2, MIR3170, LINC01007, TSPAN17, MIR4693, HYOU1,

TLR4, ADGRL3, IL6, VIRMA, MKRN1, INSIG1, ROR2, MRPL3, FMNL2, TMEM19, ZNF438, LINC01192, RCBTB1, TSPAN33, NKD2, FGFR2, TPRG1, MIR4644, FOX04, FSTL1 and CLMN, and/or

(ii) a hypomethylation of genes selected among NT5C2, NAV1, SODS, C3, UBE3A, MIR4655, MYO5C, COX6C, MIR6133, BRSK2, MIR4277, MIR4251, MN1, MIR3666, AZIN1, MIR4251, SLC37A2, FZD10, STAU2-AS1, TDRDS, USP1, ACVR2A, FBXO38, FASN, MKRN9P and PCCA-AS1,

Here, “hypermethylation of a gene” (or “hypomethylation of a gene”) shall mean a level of methylation greater (or less) than the normomethylation level of 15% of the coding sequence of the gene, leading to an increase (or repression) of the expression of this gene.

Among the 36 hypermethylated genes (list (i) above) and the 26 hypomethylated genes (list (ii) above) in endometriotic patients, the inventors have identified 15, the analysis of which, by itself, makes it possible to differentiate the endometriotic patients from the others within a studied cohort. This list consists of the following genes: CALD1, RRP1, FN1, FAM87B, TCEAL6, RPL29P2, ATP11A-AS1, DIP2C, SLCO2B1, RMI2, FBX038, ACVR2A, USP1, TDRDS and STAU2-AS1. Of course, a similar analysis on a larger cohort is likely to lead to additions or replacements in this list, without going beyond the scope of the present application. According to a particular embodiment of the invention the level of methylation is measured for at least 5 genes chosen in the group consisting of CALD1, RRP1, FN1, FAM87B, TCEAL6, RPL29P2, ATP11A-AS1, DIP2C, SLCO2B1, RMI2, FBX038, ACVR2A, USP1, TDRDS and STAU2-AS1.

For example, the method can be implemented by measuring the level of methylation of 5, 6, 7, 8, 9, 10, 11, 12, 13, 14 or 15 genes chosen in the group consisting of CALD1, RRP1, FN1, FAM87B, TCEAL6, RPL29P2, ATP11A-AS1, DIP2C, SLCO2B1, RMI2, FBX038, ACVR2A, USP1, TDRD5 and STAU2-AS1.

During the implementation of this aspect of the invention, the methylation profile of the DNA can be measured by any method described in the scientific literature.

In particular, three major molecular methods, based on enzymatic immunological or chemical detection, allow mapping of the methylated cytosines. In the context of the present invention, these different techniques can be combined with on-chip hybridization methods or high-throughput sequencing for more detailed resolution. The four techniques most commonly used are MeDIP-seq, WGBS, RRBS and 450K Bead Array. These different methods can be easily implemented by a person skilled in the art, thanks to the availability of detailed protocols in the literature, commercial kits and specialized laboratories. These different methods produce consistent results, however with varying detection sensitivities of differentially methylated regions between samples. Also, in the context of the present invention, the “normal” level of methylation for the genes analyzed must be calibrated by using the technique which will be used for measuring the level of methylation of the genes from the biological sample.

### Immunological Detection Method

The specific antibodies of methylated cytosines enable detection by immunoprecipitation, according to a method referred to as MeDIP (Methylated DNA ImmunoPrecipitation). Conventionally, the DNA is fragmented by sonication, and the most methylated fragments are those most preferentially precipitated in the presence of the antibodies, making it possible to obtain a methylation enriched fraction of the genome. In the context of the present invention, the sonication step is not indispensable, given the fragmented nature of acellular DNA. Coupled with high-throughput sequencing (MeDIP-seq), this method makes it possible to measure a local methylation density with a resolution of approximately 200 nucleotides, corresponding to the average size of the fragments, at a reasonable cost. It allows the genome to be completely covered with, however, a bias for the regions which are richest in CpG units.

### Chemical Detection Methods

The only tool which can investigate the methylation status on the scale of individual cytosine is based on bisulfite. In the presence of this chemical compound, cytosines are converted into uracil, whereas methylated cytosines are not affected. This method thus enables a reading of the methylation by analysis of the simple nucleic polymorphisms (SNP), in which a T corresponds to an unmodified cytosine and a C to a methylated cytosine on the reference genome before conversion.

The Whole-Genome Bisulfite Sequencing of the DNA (WGBS) makes it possible to access the methylation status of all the cytosines, representing the excellence of all the genomic methylation mapping methods.

RRBS (Reduced Representation Bisulfite Sequencing) is a technique derived from WGBS, based on the prior selection of the genomic regions that are rich in CpG through the use of restriction enzymes. By reducing the number of fragments to be sequenced, the cost and depth of the sequencing is greatly improved, on the same order of magnitude as MeDIP-seq.

Finally, the DNA converted with bisulfite can also be hybridized on an oligonucleotide chip, comprising specific oligonucleotides of the differentially methylated genes in the endometriotic patients.

During the implementation of the above method, the level of methylation of the genes measured makes it possible to confirm the endometriosis diagnosis, but also to make it more precise.

In particular, according to a particular embodiment of the invention, the level of methylation of the measured genes makes it possible to characterize the potential for development of endometriosis. Indeed, this potential for development is all the more important, since the methylation profile of the genes analyzed is characteristic of endometriotic patients. In particular, the potential for development will be considered as particularly important when measuring a significant or large hypermethylation of at least 7 genes selected from among CALD1, RRP1, FN1, FAM87B, TCEAL6, RPL29P2, ATP11A-AS1, DIP2C, SLCO2B1, RMI2, MIR3170, LINC01007, TSPAN17, MIR4693, HYOU1, TLR4, ADGRL3, IL6, VIRMA, MKRN1, INSIG1, ROR2, MRPL3, FMNL2, TMEM19, ZNF438, LINC01192, RCBTB1, TSPAN33, NKD2, FGFR2, TPRG1, MIR4644, FOX04, FSTL1 and CLMN, and/or a significant or large hypomethylation of at least 3 genes selected from among NT5C2, NAV1, SODS, C3, UBE3A, MIR4655, MYO5C, COX6C, MIR6133, BRSK2, MIR4277, MIR4251, MN1, MIR3666, AZIN1, MIR4251, SLC37A2, FZD10, STAU2-AS1, TDRDS, USP1, ACVR2A, FBX038, FASN, MKRN9P and PCCA-AS1. A person skilled in the art is able to define the values from which he would consider a hyper- or hypomethylation to be significant or large. By way of indicative value, hyper- or hypomethylation can be considered significant if the absolute value of the methylation differential is greater than 10, and as large if the absolute value of the methylation differential is greater than 20.

Thus, according to a particular embodiment of the invention, a large hypermethylation of the genes CALD1, RRP1, FN1, FAM87B, TCEAL6, RPL29P2, ATP11A-AS1, DIP2C, SLCO2B1 and RMI2, and a large hypomethylation of the genes FBX038, ACVR2A, USP1, TDRDS and STAU2-AS1 leads to the diagnosis of endometriosis capable of rapidly aggravating.

The present invention also relates to a set or diagnostic kit for determining the potential for development of an endometriosis, comprising the reagents for measuring the level of methylation of at least 15 genes selected among those cited in table 1 above.

According to a particular embodiment, the kit according to the invention comprises primers and/or specific probes of at least 15 genes such as defined above. In particular, the kit can comprise primers and/or specific probes for 15 genes cited in Table 1, among which 5, 6, 7, 8, 9, 10, 11, 12, 13, 14 or 15 genes are chosen in the group consisting of CALD1, RRP1, FN1, FAM87B, TCEAL6, RPL29P2, ATP11A-AS1, DIP2C, SLCO2B1, RMI2, FBX038, ACVR2A, USP1, TDRDS and STAU2-AS1.

According to a particular embodiment, the kit according to the invention comprises an oligonucleotide chip sensitive to methylation including oligonucleotides specific to at least 15 genes such as defined above. In particular, the chip can comprise specific oligonucleotides of 15 genes cited in Table 1, among which 5, 6, 7, 8, 9, 10, 11, 12, 13, 14 or 15 genes are chosen in the group consisting of CALD1, RRP1, FN1, FAM87B, TCEAL6, RPL29P2, ATP11A-AS1, DIP2C, SLCO2B1, RMI2, FBX038, ACVR2A, USP1, TDRDS and STAU2-AS1.

According to another particular embodiment, the kit according to the invention also comprises specific antibodies of methylated cytosines.

According to another particular embodiment, the kit according to the invention also comprises reagents for measuring the level of acellular DNA in the biological sample.

The present invention is further illustrated in the experimental part below, which does not limit the scope.

### EXAMPLES

### Example 1

### Measuring the Quantity of Free DNA in the Blood of Patients Having Endometriosis

### Materials and Methods

### Cohort

A group of 32 women (16 without antecedent of endometriosis and 16 with medically and/or surgically treated endometriosis antecedents) were the subject of the study below, with their consent and the agreement of the Ethics Committee of the University of Sousse (Tunisia).

The blood samples were analyzed by extraction of the free DNA and quantitative PCR in order to know the difference in concentration of circulating free DNA between the two groups)

### Evaluation of the Circulating DNA by Real-Time Quantitative PCR

### Materials

The following primers have been used:

RNP30_F:
(SEQ ID No: 1)
AGATTTGGACCTGCGAGCG
and
RNP30_R:
(SEQ ID No: 2)
GAAGCCGGGGCAACTCAC.

These primers amplify a region of 86 base pairs spanning exon 1 and intron 2 of the gene Homo sapiens ribonuclease P/MRP subunit p30, abbreviated as RPP30 (NM_006413, ENST00000371703.7).

A real-time thermal cycler (LightCycler® 480 Instrument II—Roche Life Science) was used, in accordance with the manufacturer's instructions. A “Master mix” containing SYBR™ Green (LightCycler® 480 SYBR Green I Master—Cat. No 04707516001) was used as well as a stable Taq polymerase, without enzymatic activity at ambient temperature, which is activated during the denaturation step.

### Blood Samples

The blood was sampled in DRY tubes.

Extraction of cfDNA

In order to recover the supernatant, a plurality of centrifugations was carried out in the following manner:

a first centrifugation at 1600 g for 10 minutes, then recovery of the supernatant (serum).

a second centrifugation of the serum at 3200 g for 20 minutes in a refrigerated centrifuge at 4° C. This centrifugation enabled all of the cellular debris to be removed.

Once extracted, the samples were congealed at −20° C. Calibration curves have been produced using DNA of known concentration.

The extraction of the cfDNA was monitored with internal controls coming from serum from non-invasive prenatal diagnosis patients for the diagnosis of the trisomies 13,18,21 and XY.

### Calibration Range and Internal Controls

For each PCR series, a calibration range has been produced in order to precisely assay the free DNA samples. This 8-point range (50 ng/pL to 5.10⁻⁶ ng/μL), produced by successive dilutions, made it possible to obtain a straight-line calibration, the highest and lowest concentrations of which encompass the concentrations of the samples.

The samples to be analyzed were passed at least in duplicate, and the average of the measurements was calculated.

Negative controls (Master mix only and Master mix+buffer) were carried out on each plate.

### Method for Measuring the Concentration of Free DNA

A volume of 5 μL of extracted DNA was added to 20 μL of 1×Master mix containing 0.5 μM of each primer (in triplicate). The amplification consisted of an activation of 5 minutes at 95° C. then 35 denaturation cycles at 95° C. for 10 seconds, hybridization at 59° C. for 20 seconds and elongation at 72° C. for 15 seconds, followed by a final elongation of 5 minutes at 72° C.

The calculation of the concentration was made by linear regression with a dilution scale of 10 times, in triplicate, of a known concentration of human DNA (starting from 500,000 copies/reaction to 1000 copies per reaction) and comparing the Ct (“Cycle threshold”) according to the known methods described for quantitative PCR (qPCR).

At the end of the amplification, a melting curve (Tm or “melting temperature”) has been produced in order to verify that a single product has been amplified by PCR.

The temperature is taken to 95° C., then reduced to the hybridization temperature of the primers. Then it is increased in order to separate the strands. The fluorescence is measured throughout the hybridization.

Each amplification product has a melting temperature which depends on its composition of GC (Guanine, Cytosine), the length of its nucleotide sequence, which is also influenced by the concentration of salts (MgCl₂) and by the concentration of SYBR™ Green. The results, expressed by the first derivative of the curve, show two peaks. The first peak corresponds to the amplified DNA strands, and the second to the pairing of the primers to each other.

### Results

The results of the quantification of cfDNA by PCR in real-time are given in Table 2 below.

TABLE 2
Results for the Quantification of cfDNA by real-time PCR
Endometriosis Group 1 to 16. Control group: 17 to 32.
The samples selected for performing the analysis (example
2) are indicated by a cross in the last column.

| | genomic | | ng of | |
|---|---|---|---|---|
| | copies/mL | SD | DNA/mL | Samples |
| Samples | of plasma | [%] | of plasma | selected |
| 1 | 19,325 | 0.5% | 66.64 | |
| 2 | 43,000 | 1.0% | 148.28 | |
| 3 | 248,750 | 4.2% | 857.76 | X |
| 4 | 76,750 | 0.7% | 264.66 | |
| 5 | 209,500 | 0.8% | 722.41 | X |
| 6 | 80,250 | 0.8% | 276.72 | |
| 7 | 465,000 | 1.4% | 1,603.45 | X |
| 8 | 205,750 | 1.3% | 709.48 | X |
| 9 | 78,000 | 0.8% | 268.97 | |
| 10 | 158,750 | 3.3% | 547.41 | |
| 11 | 103,000 | 1.1% | 355.17 | |
| 12 | 395,000 | 1.3% | 1362.07 | X |
| 13 | 40,750 | 0.2% | 140.52 | |
| 14 | 113,750 | 2.2% | 392.24 | |
| 15 | 79,000 | 3.0% | 272.41 | |
| 16 | 31,500 | 4.5% | 108.62 | |
| 17 | 23,750 | 0.4% | 81.90 | |
| 18 | 35,250 | 3.0% | 121.55 | |
| 19 | 228,500 | 1.5% | 787.93 | |
| 20 | 70,000 | 2.2% | 241.38 | X |
| 21 | 26,250 | 2.0% | 90.52 | |
| 22 | 26,250 | 0.0% | 90.52 | |
| 23 | 65,250 | 1.7% | 225.00 | X |
| 24 | 38,500 | 0.2% | 132.76 | |
| 25 | 61,750 | 13.0% | 212.93 | |
| 26 | 84,500 | 2.9% | 291.38 | X |
| 27 | 41,750 | 1.3% | 143.97 | |
| 28 | 69,250 | 4.3% | 238.79 | X |
| 29 | 209,750 | 4.2% | 723.28 | |
| 30 | 332,500 | 6.6% | 1146.55 | |
| 31 | 38,750 | 2.5% | 133.62 | |
| 32 | 75,000 | 0.0% | 258.62 | X |

On average, the endometriosis group contained 56% more free DNA than the control group.

Five samples from each group were chosen for carrying out a complete sequencing (“Whole Genome Sequencing”) and an analysis of the methylation profiles (methylome). The samples chosen in the endometriosis group contain, on average, four times more free DNA than the samples chosen in the control group (1051 ng/mL versus 251 ng/mL).

In the control group, samples 19, 29 and 30, exhibiting three very high values, are likely to correspond to an undiagnosed infraclinical inflammatory state. In the “endometriosis” group, one value (sample 1) is very low and three values are within the normal limits (samples 2, 13 and 16). All the women recruited in the endometriosis group had had or were having a medical treatment in progress. The normal or near normal values are likely to reflect the effectiveness of the treatment.

Following the amplification by PCR, the DNA was separated by migration over agarose gel, in order to measure the size of the DNA fragments in the cfDNA. This made it possible to observe the size of DNA fragments, ranging from 1353 to 72 pb.

### Example 2

### Analysis of the Differential Methylation Profile of the cfDNA

The cfDNA of five women from each of the two groups (see Table 2) underwent a whole genome sequencing (WGS) with, for each of the genes identified, its methylation status. This study was carried out by the ACOBIOM research platform at Montpellier.

### Materials and Methods

### Preparation of the DNA

The bisulfite treatment of the DNA was carried out according to the ROCHE protocol (KAPA Library Preparation Kit Illumina®, 07138008001), deleting the DNA fragmentation step, which serves no purpose since the circulating DNA is naturally strongly fragmented.

The ligation of the A and B adapters has been carried out according to the ROCHE protocol (SeqCap® Adapter Kit A and/or B, 07141530001), by modifying the ligation step of the adapters: 30 min at 20° C. then one night at 16° C.

Finally, the conversion to bisulfite was carried out according to the ZYMO protocol (EZ DNA Methylation-Lightning™ kit, D5030).

### DNA Sequencing

The DNA libraries were prepared according to the ROCHE kit protocol (DNA capture with the kit of the SeqCap® Epi Enrichment System, 05634261001) modifying the number of PCR cycles before capture: 14 pre-capture PCR cycles.

Before sequencing, the DNA was verified and arrayed on the PERKINELMER platform (Labchip® GX). The sequencing was carried out according to the ILLUMINA protocol on the NextSeq® platform.

### Bioinformatics Processing

### Processing Steps

The analysis of the methylation data obtained by sequencing on the Illumina platform based on the kit “Roche NimbleGen SeqCap® Epi target enrichment”, was carried out using free software and databases. The data were analyzed according to the protocol provided by Roche.

Typically, the methylation analysis followed these different steps:
(i) Quality control of sequences (software: FastQC)

Measuring and reporting the quality of the bases for each file, in order to estimate and confirm the quality of the sequencing.
(ii) Cleaning the sequences (software: Trimmomatic v 0.36)

Cleaning the Illumina sequences by following certain criteria:

deleting the poor-quality sequences

recognizing and eliminating artificial sequences (adapters+bar codes)

deleting sequences which are too short.
(iii) Aligning sequences on the reference genome (software: BSMAP v2.90)

Align each bisulfite treated sequence on the reference genome: homo-sapiens genome version GRCh37 (hg19).
(iv) Sorting and deleting PCR-generated duplicates (software: Samtools v1.8, Bamtools v2.4.1 & Picard/MarkDuplicates v2.8.1)

During the bisulfite treatment, the non-methylated C bases are transformed into T. This has the consequence that the two DNA strands are not complementary. During the PCR, the amplification of the strands will artificially generate new complementary stands that must be removed.
(v) Measuring the percentage of methylation (software: BSMAP v 2.90)

Determine the percentage methylation for each base C, for each of the files.

These results are used for the differential analysis of the methylated regions (software: methylKit R) (see the Statistical Treatment step).
(vi) Estimation of the level of efficiency of conversion by bisulfite (BSMAP v 2.90)

In order to determine the conversion efficiency with bisulfite, the number of C converted for one DNA molecule which has not been methylated is measured. The kit used contains a control sequence, Lambda phage DNA (GenBank Identifier: NC_001416), which is added to the sample. The kit also contains probes for capture of the Lambda phage.

After the alignment step, the number of C transformed into T on the specific sequences of the Lambda phage is measured. This level can be close to 100%.

### Monitoring the Sequencing Libraries

The FastQC software has been used to carry out quality controls on the data from high throughput sequencing. A report was generated for each sequencing file. The MultiQC software has grouped together these quality control reports in order to generate a summary in a single report (in html).

### Monitoring the Bisulfite Treatment

In order to determine the efficiency of the conversion, the number of converted C on the (non-methylated) DNA of the Lambda phage was measured. All the samples have been tested and the mean conversion rate to bisulfite is greater than 99.4%. These results make it possible to consider that the step of conversion to bisulfite is correctly performed.

### Results

The analysis of samples allowed 91 hypermethylated genes (Table 3) and 66 hypomethylated genes (Table 4) to be identified in endometriotic women.

TABLE 3
hypermethylated genes in endometriotic women

| | | GenBank | Methylation |
|---|---|---|---|
| Name | Description | identifier | differential |
| CALD1 | caldesmon 1 | NM_033140 | −63.7 |
| RRP1 | ribosomal RNA processing 1 | NM_003683 | −54.3 |
| FN1 | fibronectin 1 | NM_212482 | −54.3 |
| FAM87B | family with sequence similarity 87 member B | NR_103536 | −52.5 |
| TCEAL6 | transcription elongation factor A like 6 | NM_001006938 | −50.7 |
| RPL29P2 | ribosomal protein L29 pseudogene 2 | NR_002778 | −50.5 |
| ATP11A-AS1 | ATP11A antisense RNA 1 | NR_046661 | −50.0 |
| DIP2C | disco interacting protein 2 homolog C | NM_014974 | −47.0 |
| SLCO2B1 | solute carrier organic anion transporter family | NM_007256 | −46.5 |
| | member 2B1 | | |
| RMI2 | cpRecQ mediated genome instability 2 | NM_152308 | −46.4 |
| CSTF2T | cleavage stimulation factor subunit 2 tau variant | NM_015235 | −46.0 |
| LOC283177 | uncharacterized LOC283177 | NR_033852 | −44.5 |
| FXYD2 | FXYD domain containing ion transport regulator 2 | NM_001680 | −44.0 |
| ZNF33B | zinc finger protein 33B | NM_006955 | −42.2 |
| MIR3170 | microRNA 3170 | NR_036129 | −42.1 |
| LINC01007 | long intergenic non-protein coding RNA 1007 | NR_103749 | −41.4 |
| RNU6-2 | RNA, U6 small nuclear 2 | NR_125730 | −40.9 |
| REEP3 | receptor accessory protein 3 | NM_001001330 | −40.9 |
| ALG10 | ALG10, alpha-1,2-glucosyltransferase | NM_032834 | −40.8 |
| OR2AG1 | olfactory receptor family 2 subfamily AG member 1 | NM_001004489 | −40.5 |
| IQGAP2 | IQ motif containing GTPase activating protein 2 | NM_006633 | −40.2 |
| TSPAN17 | tetraspanin 17 | NM_012171 | −39.7 |
| MIR4693 | microRNA 4693 | NR_039842 | −39.5 |
| HYOU1 | hypoxia up-regulated 1 | NM_006389 | −39.0 |
| TLR4 | toll like receptor 4 | NM_138554 | −38.8 |
| LINC00689 | long intergenic non-protein coding RNA 689 | NR_024394 | −38.8 |
| PCOTH | upstream in-frame stop codon | NM_001135816 | −38.7 |
| MLN | motilin | NM_001040109 | −38.6 |
| ADGRL3 | adhesion G protein-coupled receptor L3 | NM_015236 | −37.9 |
| LINC01939 | long intergenic non-protein coding RNA 1939 | NR_110179 | −37.6 |
| ALG1L2 | upstream in-frame stop codon | NM_001136152 | −37.5 |
| IL6 | interleukin 6 | NM_000600 | −37.1 |
| PHYKPL | 5-phosphohydroxy-L-lysine phospholyase | NR_103508 | −36.8 |
| ZSWIM4 | zinc finger SWIM-type containing 4 | NM_023072 | −36.6 |
| VIRMA | vir like m6A methyltransferase associated | NM_015496 | −35.8 |
| RPS6KA2 | ribosomal protein S6 kinase A2 | NM_001006932 | −34.8 |
| FAM25A | family with sequence similarity 25 member A | NM_001146157 | −34.8 |
| MKRN1 | makorin ring finger protein 1 | NR_117084 | −34.5 |
| ADARB2 | adenosine deaminase RNA specific B2 (inactive) | NM_018702 | −34.5 |
| MGC16025 | uncharacterized LOC85009 | NR_026664 | −34.5 |
| PFKP | phosphofructokinase, platelet | NM_001242339 | −33.2 |
| FMO2 | flavin containing monooxygenase 2 | NM_001460 | −33.2 |
| INSIG1 | insulin induced gene 1 | NM_198337 | −33.1 |
| LINC00824 | long intergenic non-protein coding RNA 824 | NR_121672 | −32.7 |
| ABR | active BCR-related | NM_001256847 | −32.4 |
| ROR2 | receptor tyrosine kinase like orphan receptor 2 | NM_004560 | −32.2 |
| MRPL3 | mitochondrial ribosomal protein L3 | NM_007208 | −31.6 |
| VANGL1 | VANGL planar cell polarity protein 1 | NM_001172412 | −31.5 |
| BTD | biotinidase | NM_001281723 | −31.0 |
| AFF3 | AF4/FMR2 family member 3 | NM_002285 | −30.9 |
| DNAH9 | dynein axonemal heavy chain 9 | NM_001372 | −30.8 |
| LINC02209 | long intergenic non-protein coding RNA 2209 | NR_024473 | −30.6 |
| LOC100506422 | upstream in-frame stop codon | NM_001004352 | −30.3 |
| CAP2 | upstream in-frame stop codon | NM_006366 | −30.1 |
| FMNL2 | formin like 2 | NM_052905 | −30.1 |
| TMEM19 | transmembrane protein 19 | NM_018279 | −29.9 |
| MLLT10 | MLLT10, histone lysine methyltransferase | NM_001195626 | −29.6 |
| | DOT1L cofactor | | |
| ZNF438 | zinc finger protein 438 | NM_182755 | −29.5 |
| LINC01192 | long intergenic non-protein coding RNA 1192 | NR_033945 | −29.2 |
| SCHIP1 | schwannomin interacting protein 1 | NM_001197109 | −29.2 |
| RCBTB1 | RCC1 and BTB domain containing protein 1 | NM_018191 | −29.1 |
| LOC101929420 | uncharacterized LOC101929420 | NR_110870 | −29.1 |
| TSPAN33 | tetraspanin 33 | NM_178562 | −29.0 |
| ENGASE | endo-beta-N-acetylglucosaminidase | NM_001042573 | −28.9 |
| TMPRSS6 | transmembrane serine protease 6 | NM_153609 | −28.8 |
| NKD2 | NKD inhibitor of WNT signaling pathway 2 | NM_001271082 | −28.4 |
| IGSF11 | immunoglobulin superfamily member 11 | NM_001015887 | −28.1 |
| LOC100134317 | uncharacterized LOC100134317 | NR_029389 | −28.1 |
| FGFR2 | fibroblast growth factor receptor 2 | NR_073009 | −27.9 |
| LOC100134317 | uncharacterized LOC100134317 | NR_029389 | −27.8 |
| LOC101927972 | uncharacterized LOC101927972 | NR_125848 | −27.8 |
| TPRG1 | tumor protein p63 regulated 1 | NM_198485 | −27.8 |
| DUSP21 | dual specificity phosphatase 21 | NM_022076 | −27.7 |
| MIR4644 | microRNA 4644 | NR_039787 | −27.5 |
| FOXO4 | forkhead box O4 | NM_005938 | −27.0 |
| LTBP1 | upstream in-frame stop codon | NM_000627 | −26.8 |
| LINC01164 | long intergenic non-protein coding RNA 1164 | NR_038365 | −26.6 |
| FSTL1 | follistatin like 1 | NM_007085 | −26.4 |
| LOC100287846 | uncharacterized LOC100287846 | NR_037168 | −26.4 |
| CLMN | calmin | NM_024734 | −26.3 |
| CNIH3 | cornichon family AMPA receptor auxiliary protein 3 | NM_152495 | −26.3 |
| MICUS3 | mitochondrial calcium uptake family member 3 | NM_181723 | −26.3 |
| KRTAP19-8 | keratin associated protein 19-8 | NM_001099219 | −25.9 |
| LOC101927620 | uncharacterized LOC101927620 | NR_110062 | −25.9 |
| CLMN | calmin | NM_024734 | −25.8 |
| ARL5B | ADP ribosylation factor like GTPase 5B | NM_178815 | −25.7 |
| TMEM44-AS1 | TMEM44 antisense RNA 1 | NR_047573 | −25.7 |
| FOXO4 | forkhead box O4 | NM_001170931 | −25.7 |
| JPH3 | junctophilin 3 | NM_001271605 | −25.6 |
| FAM210B | family with sequence similarity 210 member B | NM_080821 | −25.5 |
| CLMN | calmin | NM_024734 | −25.3 |

TABLE 4
hypomethylated genes in endometriotic women

| | | GenBank | Methylation |
|---|---|---|---|
| Name | Description | identifier | differential |
| NOP56 | NOP56 ribonucleoprotein | NR_027700 | 25.1 |
| FREM2 | FRAS1 related extracellular matrix protein 2 | NM_207361 | 25.3 |
| NT5C2 | 5′-nucleotidase, cytosolic II | NM_012229 | 25.5 |
| DLGAP2 | DLG associated protein 2 | NM_004745 | 25.6 |
| NAV1 | neuron navigator 1 | NM_020443 | 25.8 |
| C22orf42 | chromosome 22 open reading frame 42 | NM_001010859 | 26.2 |
| SOD3 | superoxide dismutase 3 | NM_003102 | 26.4 |
| C3 | complement C3 | NM_000064 | 27.0 |
| PGS1 | phosphatidylglycerophosphate synthase 1 | NR_111989 | 27.5 |
| UBE3A | ubiquitin protein ligase E3A | NM_130839 | 27.7 |
| FOXL1 | forkhead box L1 | NM_005250 | 28.0 |
| SEPT-1 | septin 1 | NM_018243 | 28.1 |
| MIR4655 | microRNA 4655 | NR_039799 | 28.2 |
| TSGA13 | testis specific 13 | NM_052933 | 28.3 |
| MYO5C | myosin VC | NM_018728 | 28.6 |
| LINC00211 | long intergenic non-protein coding RNA 211 | NR_110011 | 28.8 |
| PAH | phenylalanine hydroxylase | NM_000277 | 29.1 |
| PDE3A | phosphodiesterase 3A | NM_000921 | 29.1 |
| SGIP1 | upstream in-frame stop codon | NM_032291 | 29.3 |
| LINCR-0001 | uncharacterized LINCR-0001 | NR_120604 | 29.3 |
| COX6C | cytochrome c oxidase subunit 6C | NM_004374 | 29.4 |
| MIR6133 | microRNA 6133 | NR_106749 | 30.1 |
| LOC389602 | uncharacterized LOC389602 | NM_001291913 | 30.4 |
| BRSK2 | BR serine/threonine kinase 2 | NM_001256629 | 30.6 |
| MIR4277 | microRNA 4277 | NR_036240 | 30.6 |
| RPS27A | ribosomal protein S27a | NM_001135592 | 30.8 |
| FAM133B | family with sequence similarity 133 member B | NR_109929 | 31.2 |
| THNSL2 | threonine synthase like 2 | NM_018271 | 31.2 |
| LINC01968 | long intergenic non-protein coding RNA 1968 | NR_037891 | 31.5 |
| GUCA1C | guanylate cyclase activator 1C | NM_005459 | 31.6 |
| MIR4251 | microRNA 4251 | NR_036215 | 31.9 |
| LOC101928708 | uncharacterized LOC101928708 | NR_110939 | 31.9 |
| CRAT-PLPP7 | phospholipid phosphatase 7 (inactive) | NM_032728 | 31.9 |
| CSAD | cysteine sulfinic acid decarboxylase | NM_001244705 | 32.0 |
| MN1 | MN1 proto-oncogene, transcriptional regulator | NM_002430 | 32.1 |
| CBR4 | carbonyl reductase 4 | NM_032783 | 32.5 |
| LINC02421 | long intergenic non-protein coding RNA 2421 | NR_110063 | 32.6 |
| LOC101928861 | uncharacterized LOC101928861 | NR_120513 | 33.0 |
| LOC100129203 | uncharacterized LOC100129203 | NR_110295 | 33.7 |
| FAM86EP | family with sequence similarity 86 member E | NR_024253 | 34.3 |
| TAF8 | TATA-box binding protein associated factor 8 | NM_138572 | 34.9 |
| MIR3666 | microRNA 3666 | NR_037439 | 34.9 |
| LOC100129534 | small nuclear ribonucleoprotein polypeptide N | NR_024489 | 35.3 |
| | pseudogene | | |
| ZNF496 | zinc finger protein 496 | NM_032752 | 35.8 |
| GRAMD1B | GRAM domain containing 1B | NM_001286564 | 36.2 |
| AZIN1 | antizyme inhibitor 1 | NM_148174 | 36.2 |
| FAM209B | family with sequence similarity 209 member B | NM_001013646 | 36.6 |
| MIR4251 | microRNA 4251 | NR_036215 | 36.9 |
| WDTC1 | WD and tetratricopeptide repeats 1 | NM_015023 | 37.4 |
| EPS8L1 | EPS8 like 1 | NM_133180 | 37.6 |
| DNAH5 | dynein axonemal heavy chain 5 | NM_001369 | 38.1 |
| SLC37A2 | solute carrier family 37 member 2 | NM_198277 | 38.3 |
| FASTKD1 | FAST kinase domains 1 | NR_104020 | 38.5 |
| LYZL1 | lysozyme like 1 | NM_032517 | 38.6 |
| FZD10 | frizzled class receptor 10 | NM_007197 | 39.5 |
| FAM187B | family with sequence similarity 187 member B | NM_152481 | 39.9 |
| CD81 | CD81 molecule | NM_001297649 | 40.1 |
| STAU2-AS1 | STAU2 antisense RNA 1 | NR_038406 | 40.4 |
| TDRD5 | tudor domain containing 5 | NM_001199091 | 40.5 |
| USP1 | ubiquitin specific peptidase 1 | NM_003368 | 42.3 |
| ACVR2A | activin A receptor type 2A | NM_001616 | 42.5 |
| FBXO38 | F-box protein 38 | NM_001271723 | 47.3 |
| FASN | fatty acid synthase | NM_004104 | 47.7 |
| MKRN9P | makorin ring finger protein 9, pseudogene | NR_033410 | 50.2 |
| PCCA-AS1 | PCCA antisense RNA 1 | NR_047686 | 50.7 |
| RP9 | RP9, pre-mRNA splicing factor | NM_203288 | 52.8 |

The differentially methylated genes (DMG) were then classified by ontology using the Ingenuity Pathway Analysis (IPA) software, enabling identification of the most relevant signaling and metabolic pathways, molecular networks and biological functions for a list of genes, in order to search for potential biological processes, signaling pathways and reciprocal relationships between the genes of the network.

Networks of these genes have been generated based on their connectivity. A network score was calculated on the basis of the hypergeometric distribution and calculated using Fisher's exact test (the value p<0.05 was considered significant).

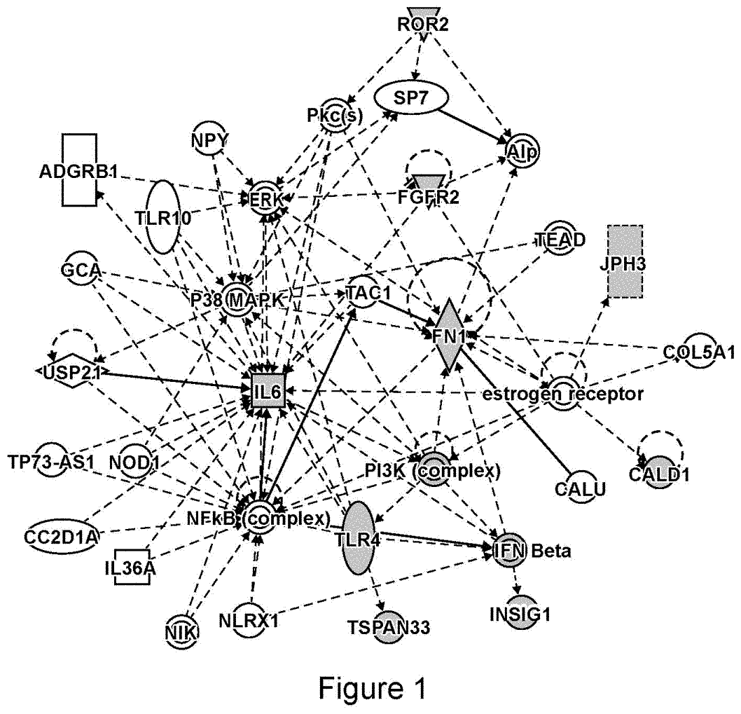

FIGS. 1 to 5 show some of the generated networks.

The first network (FIG. 1), shows that a plurality of hypermethylated genes is involved in the inflammatory and signaling diseases, as well as in interactions between cells: the importance of IL6 is noted, mainstay of inflammatory regulation and CALD 1, FN1, TLR4, JPH3 and INSIG1.

Among the hypermethylated genes, several upstream regulators of the WNTSA, IFN beta pathways and of the estrogen receptor pathways have been identified (Table 5)

TABLE 5
Main hypermethylated genes: upstream regulators
(WNT5A, IFN-beta, estrogen receptor)

| Genes: | | Methylation | | |
|---|---|---|---|---|
| Initials | Names | differential | Location | Types(s) |
| FGFR2 | Fibroblast | 27.873 | Plasma | Kinase |
| | growth factor | | membrane | |
| FN1 | Fibronectin 1 | 54.264 | Extracellular | Enzyme |
| | | | space | |
| IL6 | Interleukin 6 | 37.066 | Extracellular | Cytokine |
| | | | space | |
| ROR2 | Tyrosine kinase | 32.170 | Plasma | Kinase |
| | receptor | | membrane | |
| TLR4 | Toll like | 38.821 | Plasma | Transmembrane |
| | receptor 4 | | membrane | receptor |

Conversely, the hypomethylated gene network is enriched with genes associated with carbohydrate metabolism and with energy production (FASN, NT5C2), and with the cell cycle (UBE3A, FASN, BRSK2, SODS, PDE3A) (tables 6 to 8).

The embryonic development signature appears, in particular, in this network. ACVR2A, FZD10, CASAD, COX6C and USP1 are included.

TABLE 6
hypomethylated genes associated with carbohydrate metabolism

| Categories | Functions | p-Value | Molecules |
|---|---|---|---|
| Carbohydrate | Carbohydrate oxidation | 2.80^{E}−06 | FASN, |
| metabolism | | | NT5C2 |
| Energy production | | | |
| Carbohydrate | D-glucose oxidation | 2.00E−04 | NT5C2 |
| metabolism | | | |
| Energy production | | | |
| Carbohydrate | Glycogen incorporation | 4.79^{E}−06 | NT5C2 |
| metabolism | | | |
| Carbohydrate | D-glucose incorporation | 9.55E−03 | NT5C2 |
| metabolism, small | | | |
| molecules | | | |
| Carbohydrate | Glucose-6 phosphate | 9.55E−03 | SL37A2 |
| metabolism, | transport | | |
| molecular transport | | | |
| Carbohydrate | Monosaccharide transport | 1.00E−02 | NT5C2, |
| metabolism, | | | SLC37A2 |
| molecular transport | | | |
| Carbohydrate | Interaction with heparan | 1.19E−02 | SOD3 |
| metabolism, Drug | sulfate proteoglycan and | | |
| metabolism | collagen. Anti-oxidation | | |

TABLE 7
hypomethylated genes associated with energy production

| Categories | Functions | p-Value | Molecules |
|---|---|---|---|
| Carbohydrate | Carbohydrate oxidation | 2.80E−06 | FASN, |
| metabolism | | | NT5C2 |
| Energy production | | | |
| Carbohydrate | D-glucose oxidation | 2.00E−04 | NT5C2 |
| metabolism | | | |
| Energy production | | | |
| DNA Replication, | NADPH oxidation | 1.43E−02 | FASN |
| Recombination and | | | |
| Repair | | | |
| Energy production, | Palmitic acid oxidation | 2.61E−02 | NT5C2 |
| Lipid metabolism | | | |

TABLE 8
hypomethylated genes associated with intercellular functions

| Categories | Functions | p-Value | Molecules |
|---|---|---|---|
| Cell cycle | Senescence of lymphoma | 2.40E−03 | UBE3A |
| | cell lines | | |
| Cell cycle | G2/M arrest of the | 7.17E−03 | BRSK2 |
| | melanoma phase transition | | |
| Cell cycle | Senescence of hepatic cell | 7.17E−03 | FASN |
| | lines | | |
| Cell cycle, | Arrest of the progression of | 1.90E−02 | FASN |
| cancer | the cell cycle of tumor cells | | |
| Cell cycle | G2 phase arrest of tumor | 4.38E−02 | BRSK2, |
| | cell lines | | FASN |
| Cell cycle | G2 phase arrest of | 4.92E−02 | FASN |
| | colorectal cancer cell lines | | |

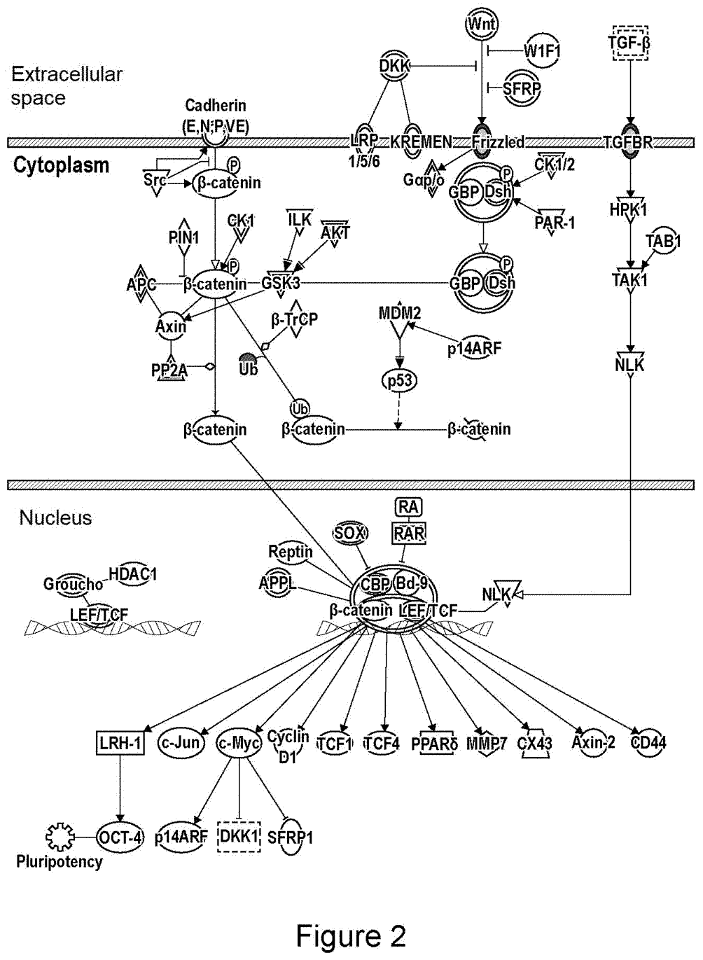

The network of FIG. 2 illustrates the transmembrane conduction pathways, in particular the involvement of the TGF-beta receptor and the frizzled transmembrane proteins (Fz), family of G protein-coupled receptors (GPCR) which have, in particular, a role in the Wnt signaling pathway.

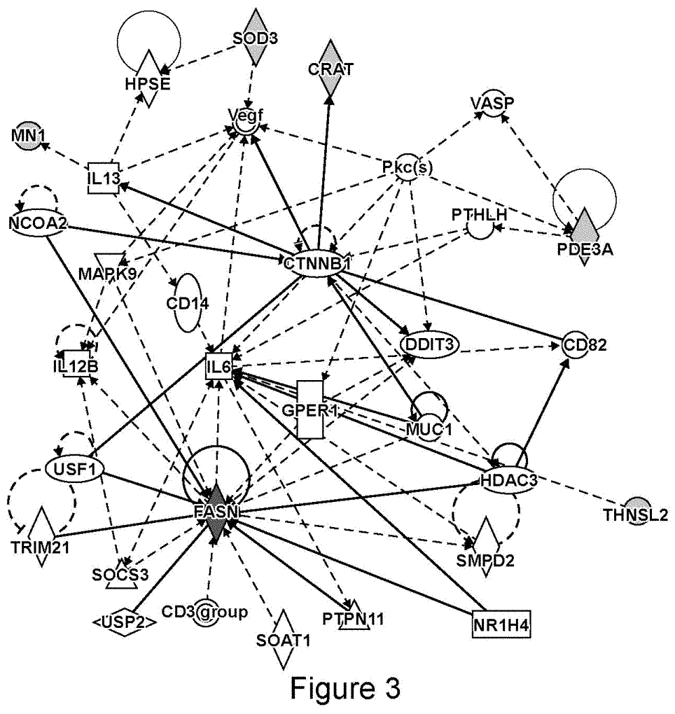

The network of FIG. 3 shows the links between the hypomethylated genes in endometriotic women, involved in cell death and survival, cellular movement and the cell cycle.

By filtering and crossing the regulatory pathways, metabolic pathways, energy production and DNA replication with its repair, a list of 15 genes only, strongly involved in endometriosis, has been selected (10 hypermethylated and 5 hypomethylated) (Table 9).

TABLE 9
15 genes (10 hypermethylated and 5 hypomethylated)
strongly involved in endometriosis

| | |
|---|---|
| Genes | Methylation differential |

Hypermethylated

| | |
|---|---|
| CALD1 | −63.726 |
| RRP1 | −54.317 |
| FN1 | −54.264 |
| FAM87B | −52.474 |
| TCEAL6 | −50.702 |
| RPL29P2 | −50.481 |
| ATP11A-AS1 | −49.984 |
| DIP2C | −47.044 |
| SLCO2B1 | −46.491 |
| RMI2 | −46.429 |

Hypomethylated

| | |
|---|---|
| FBXO38 | 47.310 |
| ACVR2A | 42.478 |
| USP1 | 42.336 |
| TDRD5 | 40.465 |
| STAU2-AS1 | 40.417 |

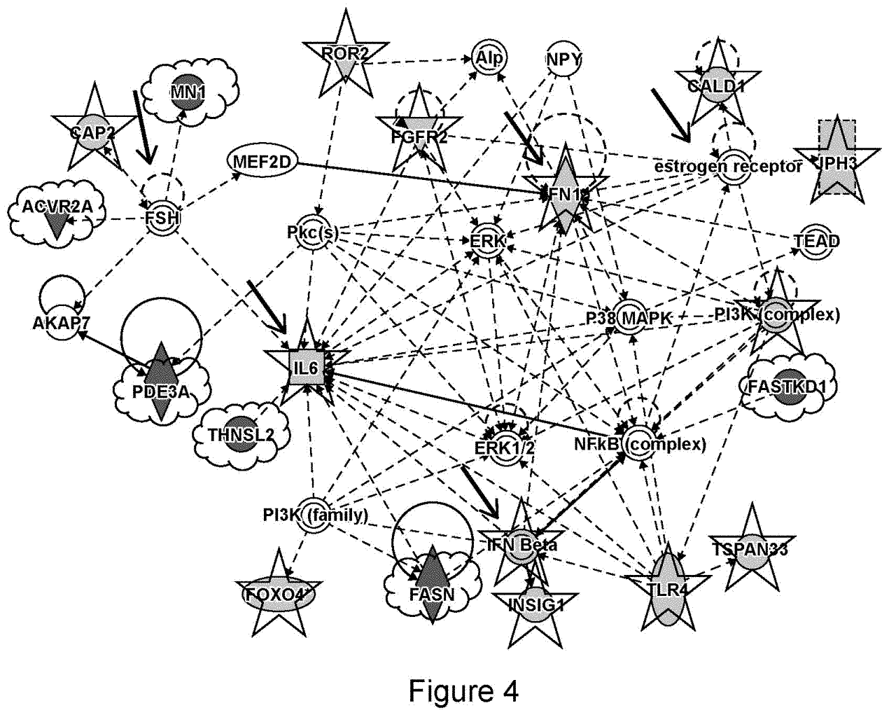

The network of FIG. 4 shows the interrelation between the hypermethylated and hypomethylated genes in connective tissue development and functional cell development. The arrows represent the essential targets.

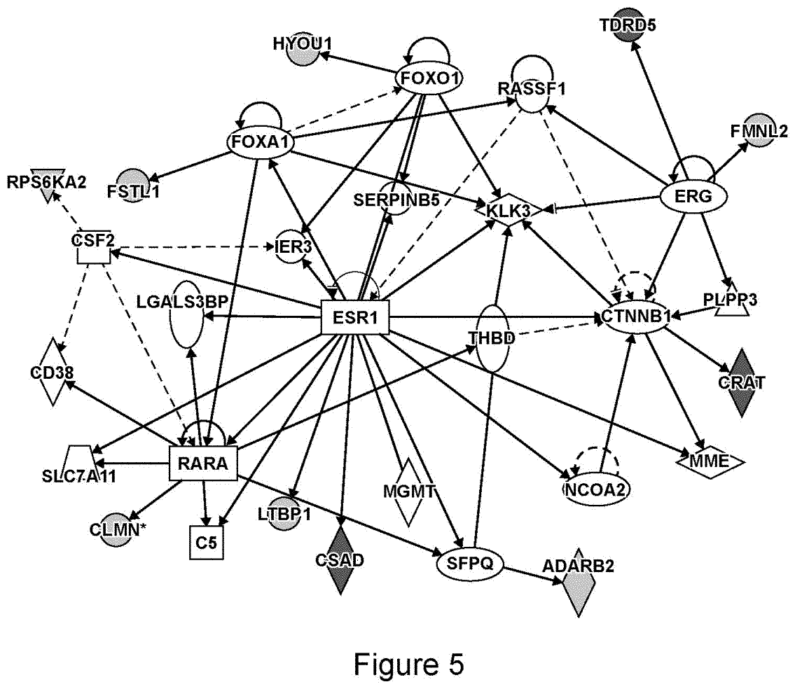

The network of FIG. 5 shows the importance of the estrogen receptor (crucial in the development of endometriosis). The selected genes (36 among the 91 hypermethylated) are indicated by a star. Though selected among the 66 hypomethylated genes (n=26) are indicated by a cloud shape.

Hence, a large part of the DMG is involved in metabolism, energy production and DNA repair and replication. In particular, it has been observed that the Wnt signaling and TGF-beta signaling pathways are significantly associated with the hypomethylated genes.

Among the 36 hypermethylated genes (CALD1, RRP1, FN1, FAM87B, TCEAL6, RPL29P2, ATP11A-AS1, DIP2C, SLCO2B1, RMI2, MIR3170, LINC01007, TSPAN17, MIR4693, HYOU1, TLR4, ADGRL3, IL6, VIRMA, MKRN1, INSIG1, ROR2, MRPL3, FMNL2, TMEM19, ZNF438, LINC01192, RCBTB1, TSPAN33, NKD2, FGFR2, TPRG1, MIR4644,

FOX04, FSTL1, CLMN) and the 26 hypomethylated genes (NT5C2, NAV1, SODS, C3, UBE3A, MIR4655, MYO5C, COX6C, MIR6133, BRSK2, MIR4277, MIR4251, MN1, MIR3666, AZIN1, MIR4251, SLC37A2, FZD10, STAU2-AS1, TDRDS, USP1, ACVR2A, FBXO38, FASN, MKRN9P, PCCA-AS1) the functions of which regulate the survival and development processes of the endometriotic cells, the inventors have therefore selected 15 genes which are particularly involved: 10 hypermethylated (CALD1, RRP1, FN1, FAM87B,TCEAL6, RPL29P2, ATP11A-AS1, DIP2C, SLCO2B1, RMI2) and 5 hypomethylated (FBX038, ACVR2A, USP1, TDRDS, STAU2-AS1). This list was selected from the raw data submitted to the networks which report the main genes which interact and their methylation status. A study on a larger cohort can, if required, lead to modifications or additions to this list.

### Discussion

The results of this serum study associate excess free DNA with a restricted panel of 15 genes, the methylation differential of which plays an essential role in the growth and development of endometriosis. To date, this is a unique non-invasive serum study, the data of which enables endometriosis to be suggested and to provide a prognosis thereon.

Endometriosis, a frequently debilitating condition, has been successively described as a hormonal disease, an immune disease, a genetic disease as well as a disease caused by exposure to environmental factors.

The economic impact of endometriosis results from the delay in diagnosing endometriosis, in particular in young women in the childbearing period who are treated too late. Laparoscopy remains the “gold standard” for diagnosis. Anatomopathological and immunohistochemical examination of the endometriotic cells has, until now, make it possible to authenticate various genes depending on the stage of endometriosis, the tissues affected and the progression of the disease. The heterogeneity of the results is due to the heterogeneity of the tissues and groups studied. Many anomalies, in particular of the cytokines (interleukin 1: IL1, mainstay of immunity, homeobox A10 (HOXA 10) and an increased expression of interleukin 6 (IL6) and of BCL2 (apoptosis factor), have been described in endometriotic cells, and the relevant genes therein have been identified. (Petracco et al. 2011; Ahn et al., 2016).

Circulating cfDNA has been proposed as a potential plasma marker of cancer, but also of minimal or mild endometriosis (Zachariah et al., 2009) with a sensitivity of 70% and a specificity of 87%.

Reflecting oxidative stress, the plasma levels of cfDNA are significantly higher in infertile women (EP2879696B1; Hazout et al., 2018).

Since inflammatory and cell stress processes are important in endometriosis states, it is not surprising to find increased levels in the plasma. However, because endometriosis can be found in asymptomatic women, the biochemical pathway for nerve stimulation by the release of certain molecules (Laux-Biehlmann et al., 2015) is not valid; this suggests that inflammation caused by the ectopic foci may not be the only cause of pelvic pain.

Indeed, endometriosis is a chronic inflammatory disease in which the sterile inflammation induced by the deposit of cyclic endometrial fragments causes epigenetic disturbances.

The epigenetic phenotypes are conferred by nuclear processes such as DNA methylation and chromatin modifications, via microRNAs and double-stranded, non-coding RNAs, which are interconnected and can operate together to establish and maintain specific states of genetic activity in normal cells (Wang et al., 2015) (Wang et al., 2016). The best understood epigenetic modification is DNA methylation at position 5 on the carbon atoms of the pyrimidine nucleus of cytosines at the cytosine-guanosine dinucleotides (CpG sites). DNA methylation converts cytosine into 5-methylcytosine which is correctly coupled with a complementary guanosine. Despite the general trend towards methylation of the CpG throughout the genome, the CpG sites of the CpG islands, and in particular those associated with gene promoters, are in general poorly methylated, which allows them to participate in active transcription of the gene.

When the CpG islands of the promoter are hypermethylated, the gene generally becomes silent. The islands are relatively stable, but reversible, and the maintenance DNA methyltransferases (DNMT) ensure epigenetic inheritance during DNA replication. Conversely, poorly methylated promoters are associated with gene activation at the transcription level and it is estimated that they make up approximately 20-30% of the human genome.

In the implementation of the invention, the serum study of the cfDNA and the methylation profile of 15 candidate genes makes it possible to confirm the presence of endometriosis.

Aberrant expression of DNA methyltransferase, (DNMT) which attaches a methyl group in position 5 of the carbon atoms of cytosines bases in the CpG island of the promoter region and silences the corresponding gene expression, has also been demonstrated in endometriosis (Naqvi et al., 2014). The accumulated evidence suggests that various epigenetic aberrations play specific roles in the pathogenesis of endometriosis (Cho et al., 2015). Recent studies have described hypermethylated or hypomethylated genes on endometriotic cells investigated by immunohistochemistry based on genes known to be involved in steroidogenesis or the expression of genes present in the endometrial tissue (Vassilopoulou et al. 2019).

In the context of blood, the degree of hypermethylation of the candidate genes (transcription blocking) reflects the intensity and/or the potential for development of the condition, possibly opposed by other active hypomethylated genes.

The present invention consists in establishing, in the bloodstream, the coexistence of an oxidative stress syndrome expressed by the serum cfDNA and the methylation status of genes involved in all the mechanisms promoting the growth and development of endometriosis: upstream regulators of cellular and molecular functions (organization, proteomics, signaling and intercellular interaction).

Of the 158 genes isolated (92 hypermethylated and 66 hypomethylated), a panel of 62 genes has been recognized in the development of endometriosis: 36 hypermethylated and 26 hypomethylated.

From this panel, after a bioinformatic analysis, a large portion of the weakly methylated genes is involved in metabolism, energy production and DNA repair and replication. The Wnt signaling and TGF-beta signaling pathways being significantly associated with the hypomethylated genes enriched with genes associated with carbohydrate metabolism and energy production (SEPT-1, FASN, NT5C2), death/survival and the cell cycle (UBE3A, FASN, BRSK2, SOD3, PDE3A). CRAT is on the 9q34.11 chromosome. This gene has also been recognized in place of PLPP7: 9q34.13, which is located on the same chromosome.

The IL6 signaling pathway is significantly associated with hypermethylated genes. The functional networks for cellular morphology, cellular assembly, inflammation and intercellular signaling are linked to the hypermethylated genes (FN1, IL6, TLR4, FGFR2, RCBTB1, IFN-beta, CALD1 and ROR2).

Moreover, FN1, CALD1 and JPH3 show a connection with the estrogen receptor.

Recent studies have shown altered levels of the expression of the gene CALD1 (coding for the protein Caldesmon) in endometriosis lesions (Meola et al., 2013). Fibronectin is involved in cell adhesion and migration processes, wound healing, blood coagulation, host defense and metastases. The gene FN1 has three regions subject to alternative splicing, with the possibility of producing 20 different transcription variants, at least one of which codes for an isoform which undergoes a proteolytic treatment. Anastellin binds to fibronectin and induces the formation of fibrils. This polymer of fibronectin, called superfibronectin, has improved adhesive properties. Anastellin and superfibronectin inhibit tumor growth, angiogenesis and metastases. JPH3 and DIP2C are expressed in the nervous system. IL6 also induces the differentiation of nerve cells.

FASN is fused with the estrogen receptor alpha (ER-alpha), SODS protects the extracellular space from the toxic effects of active derivatives of oxygen by converting superoxide radicals into hydrogen peroxide and oxygen.

A recent meta-analysis (Sapkota et al. 2017) has identified new loci associated with endometriosis, highlighting key genes involved in hormonal metabolism (including FN1 and ESR1).

In conclusion, the study presented here is the first work showing a significant increase in free DNA in the serum of women suffering from suspected endometriosis pains, associated with a panel of hypermethylated and hypomethylated genes regulating the expression and development of endometriosis.

The present invention therefore opens up prospects for early screening of endometriosis by the level of excess serum cfDNA, said screening being associated with a prognosis deduced from the methylation status of the serum cfDNA.

### Bibliography

Ahn S.Y. Singh V., and Tayade C, 2016. Biomarkers in endometriosis: challenges and opportunities. Fertil.Steril. 523-532.

Ahn Soo Hyun, Vinay Singh, and Chandrakant Tayade, 2017. Biomarkers in endometriosis: challenges and opportunities. Fertil.Steril. vol107, n° 3, 523-532.

Beckman E. N., Leonard G. L., Pintado S. O. et al., 1985. Endometriosis of the prostate. The American Journal of Surgical Pathology, vol. 9, no. 5, pp. 374-379.

Blumenkrantz M J, Gallagher N, Bashore R A, Tenckhoff H, 1981. Retrograde menstruation in women undergoing chronic peritoneal dialysis. Obstet Gynecol; 57:667-70

Cho S, Mutlu L, Grechukhina O, Taylor H S, 2015. Circulating microRNAs as potential biomarkers for endometriosis. Fertil Steril; 103:1252-60.el.

Fukunaga M, 2012. Paratesticular endometriosis in a man with a prolonged hormonal therapy for prostatic carcinoma. Pathology—Research and Practice, vol. 208, no. 1, pp. 59-61.

Halme J, Hammond M G, Hulka J F, Raj S G, Talbert L M, 1984. Retrograde menstruation in healthy women and in patients with endometriosis. Obstet Gynecol; 64:151-4.

Hazout A, Montjean D, Cassuto N G, Belloc S, Dalleac A, Tesarik J and Benkhalifa M, 2018. Free Circulating Nucleic Acids and Infertility. JGWH.

Laux-Biehlmann A, d'Hooghe T, Zollner T M, 2015. Menstruation pulls the trigger for inflammation and pain in endometriosis. Trends Pharmacol Sci; 36:270-6.

Liu K, Zhang W, Liu S, Dong B, Liu Y, 2015. Hepatic endometriosis: a rare case and review of the literature. Eur J Med Res; 20:48.

Mandel P, Metais P, 1948. Les acides nucléigues du plasma sanguin chez l'homme. [Nucleic acids in human blood plasma] CR Seance Soc Biol Ses Fil 142: 241-243.

Marsh, E. E. & Laufer, M. R., 2005. Endometriosis in premenarcheal girls who do not have an associated obstructive anomaly. Fertil. Steril. 83, 758-760.

Meola J, Hidalgo G D S, Rosa e Silva J, Costa Mendes Silva J, Paro Paz, and Rui Alberto Ferriani, 2013. Caldesmon: New Insights for Diagnosing Endometriosis. Biology of Reproduction. 88(5): 122,1-8.

Naqvi H, Ilagan Y, Krikun G, Taylor H S, 2014. Altered genome-wide methylation in endometriosis. Reprod Sci; 21:1237-43.

Nasu K, Kawano Y, Tsukamoto Y, Takano M, Takai N, Li H, Furukawa Y, Abe W, Moriyama M, Narahara H, 2011. Aberrant DNA methylation status of endometriosis: epigenetics as the pathogenesis, biomarker and therapeutic target. J Obstet Gynaecol Res. July; 37(7):683-95.

Petracco R, Grechukhina O, Popkhadze S, Massasa E, Zhou Y, Taylor H S, 2011. MicroRNA 135 regulates HOXA10 expression in endometriosis. J Clin Endocrinol Metab; 96:1925-33.

Rei C, Williams T, and M Feloney, 2018. Endometriosis in a Man as a Rare Source of Abdominal Pain: A Case Report and Review of the Literature, Case Reports in Obstetrics and Gynecology, vol. 2018, Article ID 2083121.

Sapkota Y, Steinthorsdottir V, Morris A. P., Fassbender A., Rahmioglu. R, 2017. Meta-analysis identifies five novel loci associated with endometriosis highlighting key genes involved in hormone metabolism. Nature communications.

Vassilopoulou L, Matalliotakis M, Zerviou M, atalliotaki C, Krithinakis K, Matalliotakis I, Spandidos D, Goulielmos G. N. Defining the genetic profile of endometriosis. Review 2019. Experimental and Therapeutic Medicine.

Wang Y, Li Y, Yang Z, Liu K, Wang D, 2015. Genome-wide microarray analysis of long noncoding RNAs in eutopic secretory endometrium with endometriosis. Cell Physiol Biochem; 37:2231-45.

Wang W T, Sun Y M, Huang W, He B, Zhao Y N, Chen Y Q, 2016. Genome-wide long noncoding RNA analysis identified circulating IncRNAs as novel noninvasive diagnostic biomarkers for gynecological disease. Sci Rep; 6:23343.

Zachariah R, Schmid S, Radpour R, Buerki N, Xiu-Cheng Fan A, Hahn S, Holzgreve W, Zhong X Y, 2009. Circulating cell-free DNA as a potential biomarker for minimal and mild endometriosis. RBMonline: 407-411.

1. Method of in vitro screening for endometriosis, comprising
(i) measuring the level of acellular DNA in a biological sample from an individual;
(ii) comparing the level of acellular DNA with a predetermined threshold;
(iii) if the level of acellular DNA measured in step (ii) is less than the predetermined threshold, an endometriosis diagnosis is ruled out, and if the level of acellular DNA measured in step (ii) is greater than a predetermined threshold, then the level of methylation of at least 15 genes, selected among the genes described in the table below, is measured in the acellular DNA:

| | GenBank | | GenBank | | GenBank |
|---|---|---|---|---|---|
| Name | identifier | Name | identifier | Name | identifier |
| CALD1 | NM_033140 | ROR2 | NM_004560 | MYO5C | NM_018728 |
| RRP1 | NM_003683 | MRPL3 | NM_007208 | COX6C | NM_004374 |
| FN1 | NM_212482 | FMNL2 | NM_052905 | MIR6133 | NR_106749 |
| FAM87B | NR_103536 | TMEM19 | NM_018279 | BRSK2 | NM_001256629 |
| TCEAL6 | NM_001006938 | ZNF438 | NM_182755 | MIR4277 | NR_036240 |
| RPL29P2 | NR_002778 | LINC01192 | NR_033945 | MIR4251 | NR_036215 |
| ATP11A-AS1 | NR_046661 | RCBTB1 | NM_018191 | MN1 | NM_002430 |
| DIP2C | NM_014974 | TSPAN33 | NM_178562 | MIR3666 | NR_037439 |
| SLCO2B1 | NM_007256 | NKD2 | NM_001271082 | AZIN1 | NM_148174 |
| RMI2 | NM_152308 | FGFR2 | NR_073009 | MIR4251 | NR_036215 |
| MIR3170 | NR_036129 | TPRG1 | NM_198485 | SLC37A2 | NM_198277 |
| LINC01007 | NR_103749 | MIR4644 | NR_039787 | FZD10 | NM_007197 |
| TSPAN17 | NM_012171 | FOXO4 | NM_005938 | STAU2-AS1 | NR_038406 |
| MIR4693 | NR_039842 | FSTL1 | NM_007085 | TDRD5 | NM_001199091 |
| HYOU1 | NM_006389 | CLMN | NM_024734 | USP1 | NM_003368 |
| TLR4 | NM_138554 | NT5C2 | NM_012229 | ACVR2A | NM_001616 |
| ADGRL3 | NM_015236 | NAV1 | NM_020443 | FBXO38 | NM_001271723 |
| IL6 | NM_000600 | SOD3 | NM_003102 | FASN | NM_004104 |
| VIRMA | NM_015496 | C3 | NM_000064 | MKRN9P | NR_033410 |
| MKRN1 | NR_117084 | UBE3A | NM_130839 | PCCA-AS1 | NR_047686 |
| INSIG1 | NM_198337 | MIR4655 | NR_039799 | | |

(iv) analyzing the differential methylation profile obtained in step (iii).

2. The method according to claim 1, wherein the individual is a woman.

3. The method according to claim 1, wherein the sample is a serum sample.

4. The method according to claim 1,
wherein:
(i) a hypermethylation of genes selected among CALD1, RRP1, FN1, FAM87B, TCEAL6, RPL29P2, ATP11A-AS1, DIP2C, SLCO2B1, RMI2, MIR3170, LINC01007, TSPAN17, MIR4693, HYOU1, TLR4, ADGRL3, IL6, VIRMA, MKRN1, INSIG1, ROR2, MRPL3, FMNL2, TMEM19, ZNF438, LINC01192, RCBTB1, TSPAN33, NKD2, FGFR2, TPRG1, MIR4644, FOXO4, FSTL1 and CLMN, and/or
(ii) a hypomethylation of genes selected among NT5C2, NAV1, SOD3, C3, UBE3A, MIR4655, MYO5C, COX6C, MIR6133, BRSK2, MIR4277, MIR4251, MN1, MIR3666, AZIN1, MIR4251, SLC37A2, FZD10, STAU2-AS1, TDRD5, USP1, ACVR2A, FBXO38, FASN, MKRN9P and PCCA-AS1,
are markers for endometriosis.

5. The method according to claim 1, wherein the level of methylation is measured for at least five genes chosen in the group consisting of CALD1, RRP1, FN1, FAM87B, TCEAL6, RPL29P2, ATP11A-AS1, DIP2C, SLCO2B1, RMI2, FBX038, ACVR2A, USP1, TDRD5 and STAU2-AS1.

6. The method according to claim 5, wherein the level of methylation is measured for at least 10 genes chosen in the group consisting of CALD1, RRP1, FN1, FAM87B, TCEAL6, RPL29P2, ATP11A-AS1, DIP2C, SLCO2B1, RMI2, FBX038, ACVR2A, USP1, TDRD5 and STAU2-AS1.

7. The method according to claim 6, wherein the level of methylation is measured for the genes CALD1, RRP1, FN1, FAM87B, TCEAL6, RPL29P2, ATP11A-AS1, DIP2C, SLCO2B1, RMI2, FBX038, ACVR2A, USP1, TDRD5 and STAU2-AS1.

8. The method according to claim 1, wherein the level of methylation of the measured genes enables the endometriosis diagnosis to be confirmed.

9. Endometriosis screening kit, comprising reagents for measuring the level of methylation of at least 15 genes selected among:

| | GenBank | | GenBank | | GenBank |
|---|---|---|---|---|---|
| Name | identifier | Name | identifier | Name | identifier |
| CALD1 | NM_033140 | ROR2 | NM_004560 | MYO5C | NM_018728 |
| RRP1 | NM_003683 | MRPL3 | NM_007208 | COX6C | NM_004374 |
| FN1 | NM_212482 | FMNL2 | NM_052905 | MIR6133 | NR_106749 |
| FAM87B | NR_103536 | TMEM19 | NM_018279 | BRSK2 | NM_001256629 |
| TCEAL6 | NM_001006938 | ZNF438 | NM_182755 | MIR4277 | NR_036240 |
| RPL29P2 | NR_002778 | LINC01192 | NR_033945 | MIR4251 | NR_036215 |
| ATP11A-AS1 | NR_046661 | RCBTB1 | NM_018191 | MN1 | NM_002430 |
| DIP2C | NM_014974 | TSPAN33 | NM_178562 | MIR3666 | NR_037439 |
| SLCO2B1 | NM_007256 | NKD2 | NM_001271082 | AZIN1 | NM_148174 |
| RMI2 | NM_152308 | FGFR2 | NR_073009 | MIR4251 | NR_036215 |
| MIR3170 | NR_036129 | TPRG1 | NM_198485 | SLC37A2 | NM_198277 |
| LINC01007 | NR_103749 | MIR4644 | NR_039787 | FZD10 | NM_007197 |
| TSPAN17 | NM_012171 | FOXO4 | NM_005938 | STAU2-AS1 | NR_038406 |
| MIR4693 | NR_039842 | FSTL1 | NM_007085 | TDRD5 | NM_001199091 |
| HYOU1 | NM_006389 | CLMN | NM_024734 | USP1 | NM_003368 |
| TLR4 | NM_138554 | NT5C2 | NM_012229 | ACVR2A | NM_001616 |
| ADGRL3 | NM_015236 | NAV1 | NM_020443 | FBXO38 | NM_001271723 |
| IL6 | NM_000600 | SOD3 | NM_003102 | FASN | NM_004104 |
| VIRMA | NM_015496 | C3 | NM_000064 | MKRN9P | NR_033410 |
| MKRN1 | NR_117084 | UBE3A | NM_130839 | PCCA-AS1 | NR_047686 |
| INSIG1 | NM_198337 | MIR4655 | NR_039799 | | |

10. The kit according to claim 9, further comprising reagents for measuring the level of acellular DNA in a biological sample.

11. The kit according to claim 9, comprising primers and/or probes specific to the genes
CALD1, RRP1, FN1, FAM87B, TCEAL6, RPL29P2, ATP11A-AS1, DIP2C, SLCO2B1, RMI2, MIR3170, LINC01007, TSPAN17, MIR4693, HYOU1, TLR4, ADGRL3, IL6, VIRMA, MKRN1, INSIG1, ROR2, MRPL3, FMNL2, TMEM19, ZNF438, LINC01192, RCBTB1, TSPAN33, NKD2, FGFR2, TPRG1, M1R4644, FOXO4, FSTL1 and CLMN, NT5C2, NAV1, SOD3, C3, UBE3A, MIR4655, MYO5C, COX6C, M1R6133, BRSK2, M1R4277, M1R4251, MN1, M1R3666, AZIN1, M1R4251, SLC37A2, FZD10, STAU2-AS1, TDRD5, USP1, ACVR2A, FBXO38, FASN, MKRN9P and PCCA-AS1.

12. The kit according to claim 9, comprising an oligonucleotide chip sensitive to methylation, including specific oligonucleotides specific of at least 15 genes selected among

| | GenBank | | GenBank | | GenBank |
|---|---|---|---|---|---|
| Name | identifier | Name | identifier | Name | identifier |
| CALD1 | NM_033140 | ROR2 | NM_004560 | MYO5C | NM_018728 |
| RRP1 | NM_003683 | MRPL3 | NM_007208 | COX6C | NM_004374 |
| FN1 | NM_212482 | FMNL2 | NM_052905 | MIR6133 | NR_106749 |
| FAM87B | NR_103536 | TMEM19 | NM_018279 | BRSK2 | NM_001256629 |
| TCEAL6 | NM_001006938 | ZNF438 | NM_182755 | MIR4277 | NR_036240 |
| RPL29P2 | NR_002778 | LINC01192 | NR_033945 | MIR4251 | NR_036215 |
| ATP11A-AS1 | NR_046661 | RCBTB1 | NM_018191 | MN1 | NM_002430 |
| DIP2C | NM_014974 | TSPAN33 | NM_178562 | MIR3666 | NR_037439 |
| SLCO2B1 | NM_007256 | NKD2 | NM_001271082 | AZIN1 | NM_148174 |
| RMI2 | NM_152308 | FGFR2 | NR_073009 | MIR4251 | NR_036215 |
| MIR3170 | NR_036129 | TPRG1 | NM_198485 | SLC37A2 | NM_198277 |
| LINC01007 | NR_103749 | MIR4644 | NR_039787 | FZD10 | NM_007197 |
| TSPAN17 | NM_012171 | FOXO4 | NM_005938 | STAU2-AS1 | NR_038406 |
| MIR4693 | NR_039842 | FSTL1 | NM_007085 | TDRD5 | NM_001199091 |
| HYOU1 | NM_006389 | CLMN | NM_024734 | USP1 | NM_003368 |
| TLR4 | NM_138554 | NT5C2 | NM_012229 | ACVR2A | NM_001616 |
| ADGRL3 | NM_015236 | NAV1 | NM_020443 | FBXO38 | NM_001271723 |
| IL6 | NM_000600 | SOD3 | NM_003102 | FASN | NM_004104 |
| VIRMA | NM_015496 | C3 | NM_000064 | MKRN9P | NR_033410 |
| MKRN1 | NR_117084 | UBE3A | NM_130839 | PCCA-AS1 | NR_047686 |
| INSIG1 | NM_198337 | MIR4655 | NR_039799 | | |

13. The kit according to claim 9, comprising specific antibodies for methylated cytosines.

14. The method according to claim 2, wherein the sample is a serum sample.

15. The method according to claim 2,
wherein:
(i) a hypermethylation of genes selected among CALD1, RRP1, FN1, FAM87B, TCEAL6, RPL29P2, ATP11A-AS1, DIP2C, SLCO2B1, RMI2, MIR3170, LINC01007, TSPAN17, MIR4693, HYOU1, TLR4, ADGRL3, IL6, VIRMA, MKRN1, INSIG1, ROR2, MRPL3, FMNL2, TMEM19, ZNF438, LINC01192, RCBTB1, TSPAN33, NKD2, FGFR2, TPRG1, MIR4644, FOXO4, FSTL1 and CLMN, and/or
(ii) a hypomethylation of genes selected among NT5C2, NAV1, SOD3, C3, UBE3A, MIR4655, MYO5C, COX6C, MIR6133, BRSK2, MIR4277, MIR4251, MN1, MIR3666, AZIN1, MIR4251, SLC37A2, FZD10, STAU2-AS1, TDRDS, USP1, ACVR2A, FBXO38, FASN, MKRN9P and PCCA-AS1,
are markers for endometriosis.

16. The method according to claim 3,
wherein:
(i) a hypermethylation of genes selected among CALD1, RRP1, FN1, FAM87B, TCEAL6, RPL29P2, ATP11A-AS1, DIP2C, SLCO2B1, RMI2, MIR3170, LINC01007, TSPAN17, MIR4693, HYOU1, TLR4, ADGRL3, IL6, VIRMA, MKRN1, INSIG1, ROR2, MRPL3, FMNL2, TMEM19, ZNF438, LINC01192, RCBTB1, TSPAN33, NKD2, FGFR2, TPRG1, MIR4644, FOXO4, FSTL1 and CLMN, and/or
(ii) a hypomethylation of genes selected among NT5C2, NAV1, SOD3, C3, UBE3A, MIR4655, MYO5C, COX6C, MIR6133, BRSK2, MIR4277, MIR4251, MN1, MIR3666, AZIN1, MIR4251, SLC37A2, FZD10, STAU2-AS1, TDRD5, USP1, ACVR2A, FBXO38, FASN, MKRN9P and PCCA-AS1,
are markers for endometriosis.

17. The method according to claim 2, wherein the level of methylation of the measured genes enables the endometriosis diagnosis to be confirmed.

18. The method according to claim 3, wherein the level of methylation of the measured genes enables the endometriosis diagnosis to be confirmed.

19. The method according to claim 4, wherein the level of methylation of the measured genes enables the endometriosis diagnosis to be confirmed.

20. The method according to claim 5, wherein the level of methylation of the measured genes enables the endometriosis diagnosis to be confirmed.
